(19) United States
(12) Patent Application Publication
(10) Pub. No.: US 2023/0165312 A1
(43) Pub. Date: Jun. 1, 2023

(54) AEROSOL PROVISION SYSTEM

(71) Applicant: Nicoventures Trading Limited, London (GB)

(72) Inventors: Andrew Thornton, London (GB); Patrick Moloney, London (GB); Karl Kaljura, London (GB)

(21) Appl. No.: 17/997,677

(22) PCT Filed: May 4, 2021
(86) PCT No.: PCT/GB2021/051063

§ 371 Date: Nov. 1, 2022

(30) Foreign Application Priority Data
May 4, 2020 (GB) 2006537.1

(51) Int. Cl. A24F 40/50; A24F 40/48

(52) U.S. Cl. CPC A24F 40/50; A24F 40/48

(57) ABSTRACT

A component for use in an aerosol provision system is disclosed. The component includes a region through which, in use, an aerosol from the aerosol provision system passes, the component being configured to enable selective interaction of the aerosol with a substance locatable in the region.

### PRIORITY CLAIM

The present application is a National Phase entry of PCT Application No. PCT/GB2021/051063, filed May 4, 2021, which claims priority from GB Application No. 2006537.1, filed May 4, 2020, each of which is hereby fully incorporated herein by reference.

### TECHNICAL FIELD

The present disclosure relates to a component, an aerosol provision device, an aerosol provision system, an aerosol generating system, a method of generating aerosol from a component, and aerosol provision means.

### BACKGROUND

Aerosol provision systems are known. Common systems use heaters to create an aerosol from an aerosol generating material which is then inhaled by a user. The aerosol generating material from which the aerosol is generated is consumed during use of the aerosol provision system. When an aerosol generating material is heated, the aerosol generating material may change structurally. Over time such structural changes may reduce the user experience of the aerosol provision system, by virtue of changing flavors or increasing difficulty of use as the aerosol generating material is depleted. Modern systems often use a predetermined time period of active use of a system to indicate depletion of aerosol generating material within the system.

### SUMMARY

It is desirable for aerosol provision systems to prevent heating of a depleted aerosol generating material and to prevent production of undesirable flavors and aromas.

Embodiments of the present disclosure are directed toward solving some of the above problems.

In accordance with some embodiments described herein, there is provided a component for use in an aerosol provision system, the component comprising a region through which, in use, an aerosol from the aerosol provision system passes, the component being configured to enable selective interaction of the aerosol with a substance locatable in the region.

In accordance with some embodiments described herein, there is provided an aerosol provision device comprising: a first region configured for storage of an aerosol forming medium; and, a second region configured for storage of a component comprising a substance region through which, in use, an aerosol formed from the aerosol forming medium passes, the device being configured to enable selective interaction of the aerosol with a substance locatable in the substance region.

In accordance with some embodiments described herein, there is provided an aerosol provision system comprising: a first region storing an aerosol forming medium; and, a second region storing a component comprising a substance region through which, in use, an aerosol formed from the aerosol forming medium passes, the system being configured to enable selective interaction of the aerosol with a substance locatable in the substance region.

In accordance with some embodiments described herein, there is provided an aerosol generating system configured to generate an aerosol having a maximum temperature of 400° C. comprising: a device comprising a power source and a region configured for storage of an aerosol forming substrate; and, a component, the component comprising a substance region through which aerosol passes, the component being configured to enable selective interaction of the aerosol with a substance locatable in the substance region.

In accordance with some embodiments described herein, there is provided a method of generating aerosol from a component comprising: providing a component comprising a substance region, the component being configured to enable selective interaction of an aerosol with a substance locatable in the substance region; forming an aerosol from an aerosol generating material; passing the aerosol into the component; and, selectively interacting the aerosol with a substance located in the substance region.

In accordance with some embodiments described herein, there is provided aerosol provision means comprising: a first region storing an aerosol forming means; and, a second region storing a component comprising a substance region through which, in use, an aerosol formed from the aerosol forming means passes, the aerosol provision means being configured to enable selective interaction of the aerosol with substance means locatable in the substance region.

### BRIEF DESCRIPTION OF THE DRAWINGS

The present teachings will now be described by way of example only with reference to the following figures:

FIG. 1 is a perspective view of a component according to an example.

FIG. 2 is a perspective view of a component according to an example.

FIG. 3 is an end on view of a guide element according to an example.

FIG. 4 is an end on view of a guide element according to an example.

FIG. 5 is a longitudinal cross-sectional view of an aerosol provision system according to an example.

FIG. 6 is a longitudinal cross-sectional view of an aerosol provision system according to an example.

FIG. 7 is a perspective view of a component according to an example.

While embodiments of the disclosure are susceptible to various modifications and alternative forms, specific embodiments are shown by way of example in the drawings and are herein described in detail. It should be understood, however, that the drawings and detailed description of the specific embodiments are not intended to limit the invention to the particular forms disclosed. On the contrary, the disclosure covers all modifications, equivalents and alternatives falling within the scope of the present invention as defined by the appended claims.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

The present disclosure relates to aerosol provision systems, which may also be referred to as aerosol provision systems, such as e-cigarettes. According to the present disclosure, a “non- combustible” aerosol provision system is one where a constituent aerosolizable material of the aerosol provision system (or component thereof) is not combusted or burned in order to facilitate delivery to a user. Throughout the following description the term “e-cigarette” or “electronic cigarette” may sometimes be used, but it will be appreciated this term may be used interchangeably with aerosol provision system/device and electronic aerosol provision system/device. Furthermore, and as is common in the technical field, the terms “aerosol” and “vapor”, and related terms such as “vaporize”, “volatilize” and “aerosolize”, may generally be used interchangeably.

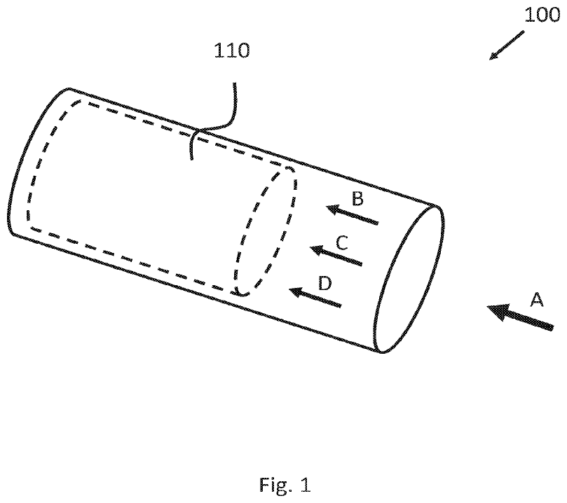

In the example of FIG. 1, a component 100 for use in an aerosol provision system is shown. In use the component 100 may be inserted into, or connected to, an aerosol provision system. The aerosol provision system may produce an aerosol. The component 100 comprises a region 110 through which, in use, an aerosol from the aerosol provision system may pass. The component 100 is configured to enable selective interaction of the aerosol with a substance locatable in the region 110.

In the example shown in FIG. 1, the aerosol provided by an aerosol provision system is shown by arrow A. The component 100 is used in the aerosol provision system downstream of an aerosol generating portion of the aerosol provision system. As such, the aerosol flow is into the component 100. This main aerosol flow is indicated by arrow A in FIG. 1. The main aerosol flow A may be divided into smaller aerosol flows which together form the main aerosol flow A. These smaller aerosol flows are represented by arrows B, C, D. These smaller aerosol flows B, C, D are incident on the region 110. The component 100 may be configured to enable selective interaction of the aerosol with a substance locatable in the region 110. In an example, the component 100 may be configured to enable selection of one or two of the smaller aerosol flows B, C, D to be incident on the region 110. In such a way, the component 100 enables selective interaction of the main aerosol flow A with a substance locatable in the region 110.

The substance may be an active substance or a flavor or both. The flavor may be olfactory or the like. The active substance may be e.g. caffeine or the like.

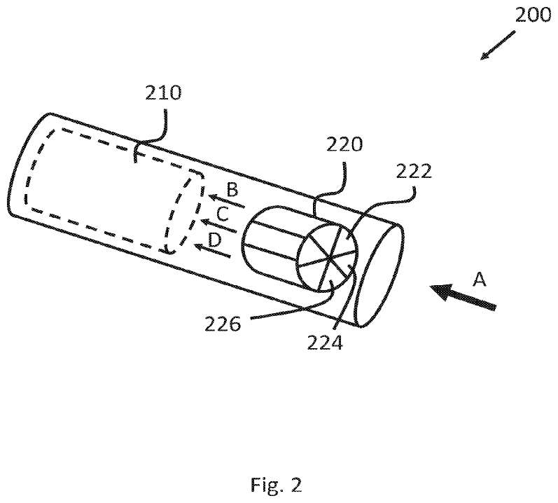

In the example of FIG. 2, an example of a component 200 is shown. The component 200 has a region 210 and a guide element 220. The guide element 220 is arranged to controllably guide aerosol through the region 210. In the specific example shown, the guide element 220 has a series of portions. In FIG. 2, the guide element has 220 is shaped in a generally cylindrical form and has 6 segments which run along the length of the cylinder of the guide element 220. Three of the segments 222, 224, 226 are specifically shown. As the aerosol A flows into the component 200, the aerosol A may, for example, pass through first segment 222, so as to form smaller aerosol flow B downstream of the guide element 220. In another example, aerosol A may pass through second segment 224 and form smaller aerosol flow C downstream of the guide element 220. In yet another example, aerosol A may pass through third segment 226 and form smaller aerosol flow D downstream of the guide element 220.

In an example, the guide element 220 may be a controlling element or the like to control (e.g. block or allow) passage of the aerosol through portions of the region 210. The guide element 220 may be a filter, or a series of filters, or a valve, or a series of valves. The guide element 220 may have a series of openable and closable doors to allow and prevent passage of aerosol through a particular door.

The aerosol A is prevented from passing through some of the guide element 220 and allowed to pass through other parts of the guide element 220. The aerosol A is therefore controlled as to which portion of the region 210 the aerosol flows. Though the term “smaller aerosol flow” is used to refer to arrows B, C, D, the total aerosol into and out of the component 200 will be substantially the same, as aerosol will not in the main be lost or contained within the component 220. As such, “smaller aerosol flow” refers to the aerosol flowing through a smaller area downstream of the guide element 220 than the larger aerosol flow which enters the component 220.

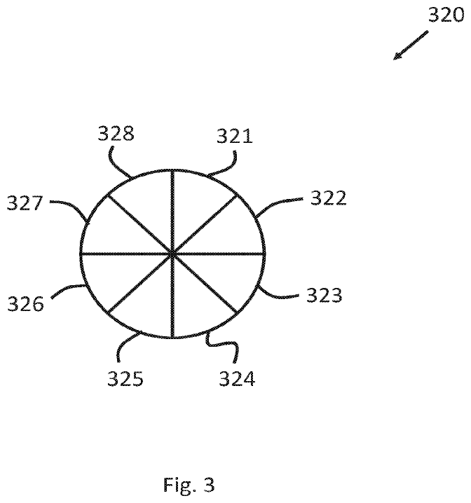

In the example of FIG. 3, an example of a guide element 320 is shown. The guide element 320 is shown end on. The guide element 320 has a plurality of sections which are separated from one another. There are eight sections 321, 322, 323, 324, 325, 326, 327, 328 shown in the Figure. The sections 321, 322, 323, 324, 325, 326, 327, 328 may be separated from one another by a frame or similar structure. The frame is advantageously impermeable to aerosol so as to ensure that aerosol that enters a particular section flows through that section and is prevented from passing into another section. In this way, the guide element 320 may control the passage of aerosol through the guide element 320.

When the guide element 320 is arranged in the component, the aerosol may be controllably forced down one (or more) of the sections 321, 322, 323, 324, 325, 326, 327, 328 of the guide element 320 and therefore into corresponding sections of the region in the component. The guide element 320 may be adjacent to, or substantially adjacent to, a substance (e.g. a flavor component or active substance) in the region. This arrangement increases the likelihood of the aerosol passing through a corresponding section of the substance.

By broadly controlling the portion of the substance through which the aerosol flows, a number of advantages are provided. As aerosol flows through the substance, the heat and moisture of the aerosol may structurally degrade the substance. Therefore, after repeated use, the substance may become structurally degraded such that the aerosol may no longer pass through the substance.

By controlling the portion of the substance through which the aerosol flows, the present device may ensure only a portion of the substance structurally degrades prior to changing the portion through which the aerosol flows.

Furthermore, the aerosol can be prevented from flowing through a depleted portion of the substance which has been found to produce undesirable compounds which are entrained in the aerosol. As such, user experience may be improved. The amount of depletion of a particular portion of the substance may be detected by a sensor and controller pair or the like. The depletion may be timed based on time of use or number of puffs or the like. Depletion may be detected by virtue of analysis of compounds being entrained in the aerosol. Once depletion is detected or a predetermined time limit is expended or even when a change of section is simply desired, the guide element 320 may be controlled to force aerosol along a different section 321, 322, 323, 324, 325, 326, 327, 328 and therefore through a different section of the substance.

The substance may contain a plurality of substance compounds to provide compounds to be entrained in the aerosol. Different sections of the substance may therefore provide different compounds for being entrained. As such, the guide element 320 may force aerosol along a first section 321 to provide a first substance compound (or compounds) to the aerosol. The guide element 320 may subsequently force aerosol along a second section 322 to provide a second substance compound (or compounds) to the aerosol. The substances may be e.g. any of tobacco, nicotine-containing, menthol, glycol, caffeine or other active substances, or the like.

Therefore, portions of the guide element 320 may for example open and close to allow passage of aerosol. The guide element 320, additionally or alternatively, may be arranged to be controllably movable. Movement of the guide element 320 may allow for the flow path of the aerosol to be controllably varied. Movement of the guide element 320 may allow for a singular open section to remain open and move so as to vary the flow path of the aerosol.

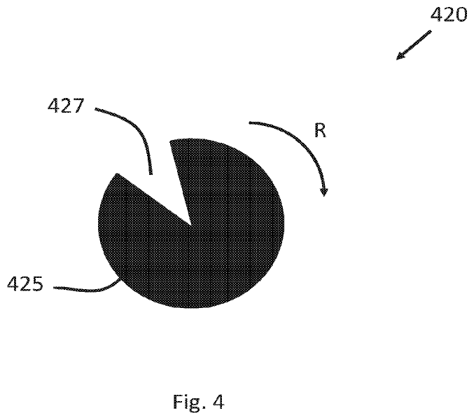

In the example of FIG. 4, an example of a guide element 420 is shown. The guide element 420 shown varies from the guide element 320 shown in FIG. 3. The guide element 420 shown has an open section 427 through which aerosol may pass and a closed section 425 through which aerosol is prevented from passing. The guide element 420 therefore only allows passage of aerosol through the open section 427. The guide element 420 may be movable to move the position of the open section 427 so as to correspondingly affect passage of the aerosol.

In an example, and as the guide element 420 shown is circular (note this is clearly not a limitation on the guide element disclosed herein) the guide element 420 may rotate in the direction shown by arrow R. Rotation of the guide element 420 moves the open section 427 so that aerosol can be forced through a different location and through a different portion of the substance in the component. This rotation may be substantially around an axis parallel to the longitudinal axial direction of the component. This may be a rotation substantially around the central axis of the guide element 420.

As noted above, the movement of the guide element 420 may be automatic (via e.g. a sensor and controller arrangement) or manual via a user input or the like. The guide element 420 may be shaped so as to correspond to the shape of the housing of the component. In this way, aerosol may be prevented from passing between the housing of the component and the guide element 420. This provides a greater level of control over the flow path of the aerosol.

The guide element may be formed of a plurality of movable elements. Turning back to FIG. 3, each section 321, 322, 323, 324, 325, 326, 327, 328 may have e.g. a respective closably openable door for controlling aerosol flow through the section 321, 322, 323, 324, 325, 326, 327, 328. These doors may be arranged, in use, to separately (or in groups) move from a respective first position to a respective second position. The respective first positions may allow aerosol to flow past the movable element. The respective second positions may prevent aerosol from flowing past the movable element. These movable elements may be doors, a series of windows, valves or actuatable switches or the like.

The controlled movement of these elements may be as a result of a sensor and controller arrangement or via user input as described above. The plurality of movable elements may be relatively movable with respect to one another. This enables selective movement of individual elements so as to controllably open individual sections 321, 322, 323, 324, 325, 326, 327, 328 of the guide element 320.

In an example, the component comprises a controller. The controller is arranged to receive a signal relating to movement of the guide element 320 and arranged to control movement of the guide element 320. The signal that is received by the controller may be provided by a sensor which may or may not be part of the component. The sensor may be part of the wider aerosol provision system with which the component is used.

The sensor may detect puffs on the system and indicate to the controller to move the guide element 320 (or some portion thereof, e.g. a door to a section) after a predetermined number of puffs. This may ensure a particular substance section is not used (i.e. does not have aerosol sent through it) after or nearing depletion. Alternatively or additionally, the sensor may detect pressure changes. As the section of the substance is depleted, structural degradation may lead to flow pressure increasing as the aerosol is increasingly prevented from flowing through that section. At a predetermined pressure, the sensor may provide a signal to the controller which in turn moves the guide element 320. Alternatively or additionally, the user may provide an indication to the controller to move the guide element 320. This may be as the user desires a change in the substance entrained in the aerosol (particularly when the substance contains a plurality of substance compounds in different sections). The user may interact with the controller via an electrical interface such as a GUI or the like, or via a mechanical interface such as a button or the like.

The controller may be connected to a movement mechanism for providing movement to the guide element 320 or portion thereof. The movement mechanism may be part of the guide element 320 or the component. The phrase “the controller moves the guide element” is intended to mean, the controller controls the movement of the guide element in some manner, such as sending a signal to a mechanical element which in turn effects the movement of the guide element.

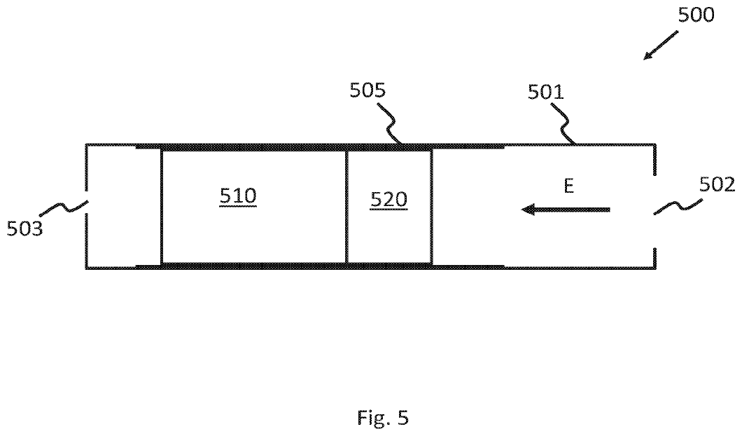

In the example of FIG. 5, an example of a portion of an aerosol provision system 500 is shown. The example of the portion of the aerosol provision system 500 is shown in a longitudinal cross-section view. The aerosol provision system 500 has a housing 501 having an inlet 502 and an outlet 503. An aerosol may enter the portion of the aerosol provision system 500 shown through the inlet 502. The incoming aerosol is shown by arrow E. The aerosol provision system 500 has a component 505 arranged within the aerosol provision system 500. The component 505 has a substance region 510, which in use contains a substance. The component 505 has a guide element 520 for controllably guiding aerosol through the substance region 510. The component 505 fits snugly in the housing 501 of the aerosol provision system 500. This prevents aerosol E flowing between the housing 501 and the component 505. The substance region 510 is shown adjacent to the guide element 520 so that the guide element 520 efficiently controls aerosol flow into particular sections of the substance in the substance region 510. The guide element 520 fits snugly in the component 505 to prevent aerosol E flowing between the component 505 and the guide element 520.

The arrangement shown in FIG. 5 enables a good level of control over the path that the aerosol takes through the component 505. This improvement in said control leads to an improvement in the user experience of the system 500 for the various reasons listed earlier. The aerosol provision system 500 may further comprise a cartomizer, atomizer or the like (not shown) for providing an aerosol. The component 505 may, as described above, further comprise a controller, a sensor, and a movement mechanism or the like (all not shown). The outlet 503 may be a mouthpiece on which a user may inhale.

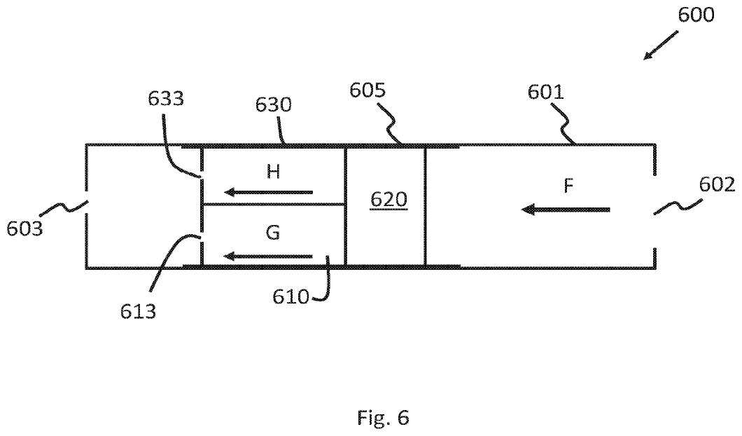

In the example of FIG. 6, an example of a portion of an aerosol provision system 600 is shown. The aerosol provision system 600 differs from the aerosol provision system 500 shown in the example of FIG. 5. The aerosol provision system 600 has a housing 601 with an inlet 602 and an outlet 603 with a component 605 having a guide element 620. The aerosol provision system 600 has a substance region 610 which in use contains a substance. The aerosol provision system 600 also has a free channel 630 which does not contain a substance in use. The aerosol flowing through the aerosol provision system 600 is shown by arrow F.

In an example, the guide element 620 of the aerosol provision system 600 may enable selective interaction of the aerosol F with a substance locatable in the substance region, indicated by arrow G. The guide element 620 may also enable selective interaction of the aerosol F not with a substance, by passing the aerosol F through the channel 630, indicated by arrow H. The channel 630 does not contain a substance.

By controlling the guide element 620, the user may optionally add a substance to the aerosol F that is passing through the aerosol provision system 600. The user may instead optionally not add a substance and so be provided with only the aerosol for inhalation. The guide element 620 may have a series of movable elements, one or more of which move to allow passage of aerosol through a portion of the substance region 610 and one or more of which move to allow passage of aerosol through a portion of the channel 630. The aerosol provision system 600 may also have movable elements over openings 633, 613 to prevent aerosol flowing through the channel 630 entering the substance region 610 through the opening 613 to the substance region. Furthermore, inhalation of the user encourages the airflow through either the substance region 610 or the channel 630 to flow out of the outlet 603 rather than back through either opening 613, 633.

To maintain user experience, the pressure drop across the channel 630 and the substance region 610, which in use contains a substance, should be similar. The user should not need to inhale much harder on the aerosol provision system 600 when passing the aerosol F through the substance in the substance region 610. Therefore, the arrangement of the substance in the substance region 610 should be such as to not create a significant pressure drop between the channel with substance 610, and the channel without 630.

The guide element 620 of component 605 in FIG. 6, may be present outside of the component 605. In that the guide element 620 may be part of an adjacent section of the aerosol provision system 600 to the component 605. In this example, the component 605 may have two channels, airflow through which may be controllable via the guide element 620 of the aerosol provision system 600.

The guide element of any of the examples described herein may be formed at least in part of a smart material. The term “smart material” is used herein to refer to a material which may change structure in light of a particular stimulus. A smart material may also be referred to by the terms “intelligent materials”, “responsive materials” or “designed materials”. Such a stimulus may be an electrical change, a thermal change, a pressure change, magnetic field change, light change, pH change, etc. A smart material changes shape more so than normal material which, of course, changes to some (more limited) extent structurally in light of a thermal change or a pressure change. A piezoelectric material may be an example of a smart material. Such a smart material may be used so that the guide element may be moved, or manipulated to move, via a stimulus rather than moving the guide element via, e.g. a movement mechanism. This may lead to fewer components in the aerosol provision system and therefore an extended lifetime of the system.

In an example, the substance region may not contain a substance in a specific portion or portions. In this way, the system may be arranged so that the aerosol flows through a corresponding portion of the guide element and the substance region, such that the aerosol does not entrain compounds from the substance in the substance region. In this way, a user preferring only an aerosol without additional substances may be provided with such.

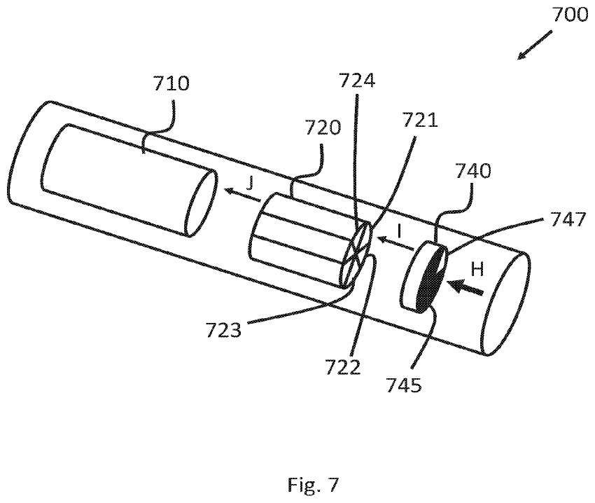

In the example of FIG. 7, an example of a portion of an aerosol provision system 700 is shown. The aerosol provision system 700 differs from previously described aerosol provision systems 500, 600 shown in the examples of FIGS. 5 and 6. The aerosol provision system 700 has two guide elements 720, 740. The aerosol provision system 700 has a substance region 710 which in use contains a substance. The aerosol flowing through the aerosol provision system 700 is shown by arrow H.

The first guide element 720 shown has an open section 747 through which aerosol may pass and a closed section 745 through which aerosol is prevented from passing. The first guide element 720 shown in FIG. 7 is similar to the guide element 420 shown and described with reference to FIG. 4. As the aerosol H is incident on the first guide element 720, the aerosol is forced through the open section 747, to form aerosol I. Having passed through the open section 747, the aerosol H is directionalized into aerosol I, which has a controllable direction.

The aerosol I is then incident on the second guide element 720. The second guide element 720 is similar to the guide element 320 shown and described with reference to FIG. 3. The second guide element 720 has a plurality of sections which are separated from one another. There are six sections shown in FIG. 7. The sections may be separated from one another by a frame or similar structure. Aerosol I may be controlled to pass through one or more of the sections. The number of sections through which the aerosol I passes can be controlled by the relative sizes of the open section 747 of the first guide element 740 and the sections in the second guide element 720. Similarly, openable elements may be used as described earlier to prevent or allow aerosol I to pass through sections of the second guide element 720. The aerosol that passes through the guide element 720 is indicated by arrow J. This aerosol is then incident on the substance region 710, which in use contains a substance.

As mentioned above, there are advantages to control on the aerosols I, J by arranging the first guide element 740 adjacent to the second guide element 740 and the second guide element 720 adjacent to the substance region 710. This assists in preventing the aerosol from being channeled by one guide element only to then bleed outwardly from the channel in which the aerosol is currently in. This can be advantageously avoided by the use of adjacent elements.

To explain this further, if there is a reasonable distance between the first guide element 740 and the second guide element 720 and aerosol may be channeled via the open section 747 so as to pass through e.g. two specific sections of the second guide element 720. In an example, the aerosol H is channeled through open section 747 to form aerosol I which was intended to pass through specific intended sections 721, 722. Distance between the first guide element 740 and the second guide element 720 however means the aerosol I spreads out between leaving the open section 747 and reaching the second guide element 720. As a result, the aerosol I passes through specific intended sections 721, 722 as well as unintended sections 723 and 724. This could lead to undesirable portions of the substance in the substance region being entrained in the aerosol providing an aerosol for inhalation not desired by the user.

The elements shown in FIG. 7 may be contained entirely, or partially, within a component of the type described earlier. The aerosol may be provided by the heating of an aerosol generating material or the like which may occur in a heating section of an aerosol provision system. The section for generating the aerosol is in fluid communication with the components shown in FIG. 7 such that an aerosol can be provided to the guide elements and controllably passed through the flavor region.

In an example of the present disclosure, the guide element may be contained within an aerosol provision device or system rather than the component. The component may then be inserted into the aerosol provision device prior to use so that the guide element controls the flow of aerosol through the component. In this example, the component may be inserted into the aerosol provision device so as to abut the guide element. Advantages of this arrangement relate to the greater control over the aerosol flow. This relates to the level of precision with which the aerosol flow can be forced through the substance region.

In such an example, the aerosol provision device may also comprise a controller to receive a signal relating to movement of the guide element. The signal may be provided by a user or from a sensor or the like. The sensor may be arranged in the aerosol provision device or in the component, and be connected to the controller. In any example, the controller may be connected electrically, or wirelessly, to the sensor.

The aerosol provision device may have a region for storing an aerosol forming medium which in use stores an aerosol forming medium. The aerosol, the flow of which is controlled by the guide element, may be formed from the aerosol forming medium by a cartomizer or atomizer or the like.

In a specific example of the present invention, there is provided an aerosol generating system configured to generate an aerosol having a maximum temperature of 400° C. The maximum temperature of 400° C. may prevent combustion of the substance in the substance region. Therefore, there is disclosed a system with selective diversion of aerosol having a maximum temperature of 400° C. The selective diversion being provided, in an example, by an element formed of a smart material.

Any of the disclosed aerosol provision systems may have control circuitry arranged to control the heating to produce an aerosol and/or receive signals from sensors or from the user and/or to control movement of the guide element (or portions thereof) or the like. The controller or the control circuitry may be connected to a database for determining when certain predetermined values are exceeded or are outside of predetermined ranges. This may lead to controlling movement of the guide element or the like to control flow of the aerosol through the aerosol provision system.

In some embodiments, the non-combustible aerosol provision system is an electronic cigarette, also known as a vaping device or electronic nicotine delivery system (END), although it is noted that the presence of nicotine in the aerosolizable material is not a requirement.

In some embodiments, the non-combustible aerosol provision system is a tobacco heating system, also known as a heat-not-burn system.

In some embodiments, the non-combustible aerosol provision system is a hybrid system to generate aerosol using a combination of aerosolizable materials, one or a plurality of which may be heated. Each of the aerosolizable materials may be, for example, in the form of a solid, liquid or gel and may or may not contain nicotine. In some embodiments, the hybrid system comprises a liquid or gel aerosolizable material and a solid aerosolizable material. The solid aerosolizable material may comprise, for example, tobacco or a non-tobacco product.

Typically, the non-combustible aerosol provision system may comprise a non-combustible aerosol provision device and an article for use with the non-combustible aerosol provision device. However, it is envisaged that articles which themselves comprise a means for powering an aerosol generating component may themselves form the non-combustible aerosol provision system.

In some embodiments, the non-combustible aerosol provision device may comprise a power source and a controller. The power source may, for example, be an electric power source.

In some embodiments, the article for use with the non-combustible aerosol provision device may comprise an aerosolizable material, an aerosol generating component, an aerosol generating area, a mouthpiece, and/or an area for receiving aerosolizable material.

In some embodiments, the aerosol generating component is a heater capable of interacting with the aerosolizable material so as to release one or more volatiles from the aerosolizable material to form an aerosol.

In some embodiments, the substance to be delivered may be an aerosolizable material. Aerosolizable material, which also may be referred to herein as aerosol generating material, is material that is capable of generating aerosol, for example when heated, irradiated or energized in any other way. Aerosolizable material may, for example, be in the form of a solid, liquid or gel which may or may not contain nicotine and/or flavorants. In some embodiments, the aerosolizable material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosolizable material may for example comprise from about 50 wt %, 60 wt % or 70 wt % of amorphous solid, to about 90 wt %, 95 wt % or 100 wt % of amorphous solid.

The aerosolizable material may comprise one or more active constituents, one or more carrier constituents and optionally one or more other functional constituents.

The active constituent may comprise one or more physiologically and/or olfactory active constituents which are included in the aerosolizable material in order to achieve a physiological and/or olfactory response in the user. The active constituent may for example be selected from nutraceuticals, nootropics, and psychoactives. The active constituent may be naturally occurring or synthetically obtained. The active constituent may comprise for example nicotine, caffeine, taurine, or any other suitable constituent. The active constituent may comprise a constituent, derivative or extract of tobacco or of another botanical. In some embodiments, the active constituent is a physiologically active constituent and may be selected from nicotine, nicotine salts (e.g. nicotine ditartrate/nicotine bitartrate), nicotine-free tobacco substitutes, other alkaloids such as caffeine.

In some embodiments, the aerosolizable material comprises one or more cannabinoid compounds selected from the group consisting of: cannabidiol (CBD), tetrahydrocannabinol (THC), tetrahydrocannabinolic acid (THCA), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) and cannabielsoin (CBE), cannabicitran (CBT).

The aerosolizable material may comprise one or more cannabinoid compounds selected from the group consisting of cannabidiol (CBD) and THC (tetrahydrocannabinol).

The aerosolizable material may comprise cannabidiol (CBD).

The aerosolizable material may comprise nicotine and cannabidiol (CBD).

The aerosolizable material may comprise nicotine, cannabidiol (CBD), and THC (tetrahydrocannabinol).

In some embodiments, the active constituent is an olfactory active constituent and may be selected from a “flavor” and/or “flavorant” which, where local regulations permit, may be used to create a desired taste, aroma or other somatosensorial sensation in a product for adult consumers. In some instances such constituents may be referred to as flavors, flavorants, cooling agents, heating agents, or sweetening agents. They may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gasone or more of extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

In some embodiments, the flavor comprises menthol, spearmint and/or peppermint. In some embodiments, the flavor comprises flavor components of cucumber, blueberry, citrus fruits and/or redberry. In some embodiments, the flavor comprises eugenol. In some embodiments, the flavor comprises flavor components extracted from tobacco. In some embodiments, the flavor may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucalyptol, WS-3.

The carrier constituent may comprise one or more constituents capable of forming an aerosol. In some embodiments, the carrier constituent may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

In some embodiments, the one or more constituents capable of forming an aerosol comprises one or more polyhydric alcohols, such as propylene glycol, triethylene glycol, 1 ,3-butanediol and glycerin; esters of polyhydric alcohols, such as glycerol mono-, di- or triacetate; and/or aliphatic esters of mono-, di- or polycarboxylic acids, such as dimethyl dodecanedioate and dimethyl tetradecanedioate.

The one or more other functional constituents may comprise one or more of pH regulators, coloring agents, preservatives, binders, fillers, stabilizers, and/or antioxidants.

The aerosolizable material may comprise an acid. The acid may be an organic acid. In some of these embodiments, the acid may be at least one of a monoprotic acid, a diprotic acid and a triprotic acid. In some such embodiments, the acid may contain at least one carboxyl functional group. In some such embodiments, the acid may be at least one of an alpha-hydroxy acid, carboxylic acid, dicarboxylic acid, tricarboxylic acid and keto acid. In some such embodiments, the acid may be an alpha-keto acid.

In some such embodiments, the acid may be at least one of succinic acid, lactic acid, benzoic acid, citric acid, tartaric acid, fumaric acid, levulinic acid, acetic acid, malic acid, formic acid, sorbic acid, benzoic acid, propanoic and pyruvic acid.

Suitably the acid is lactic acid. In other embodiments, the acid is benzoic acid. In other embodiments the acid may be an inorganic acid. In some of these embodiments the acid may be a mineral acid. In some such embodiments, the acid may be at least one of sulphuric acid, hydrochloric acid, boric acid and phosphoric acid. In some embodiments, the acid is levulinic acid.

In some embodiments, the aerosolizable material comprises a gelling agent. The gelling agent may comprise one or more compounds selected from cellulosic gelling agents, non-cellulosic gelling agents, guar gum, acacia gum and mixtures thereof.

In some embodiments, the cellulosic gelling agent is selected from the group consisting of: hydroxymethyl cellulose, hydroxyethyl cellulose, hydroxypropyl cellulose, carboxymethylcellulose (CMC), hydroxypropyl methylcellulose (HPMC), methyl cellulose, ethyl cellulose, cellulose acetate (CA), cellulose acetate butyrate (CAB), cellulose acetate propionate (CAP) and combinations thereof.

In some embodiments, the gelling agent comprises (or is) one or more of hydroxyethyl cellulose, hydroxypropyl cellulose, hydroxypropyl methylcellulose (HPMC), carboxymethylcellulose, guar gum, or acacia gum.

In some embodiments, the gelling agent comprises (or is) one or more non-cellulosic gelling agents, including, but not limited to, agar, xanthan gum, gum Arabic, guar gum, locust bean gum, pectin, carrageenan, starch, alginate, and combinations thereof. In preferred embodiments, the non-cellulose based gelling agent is alginate or agar.

In certain embodiments, the aerosolizable material comprises a gelling agent comprising a cellulosic gelling agent and/or a non-cellulosic gelling agent, an active substance and an acid.

In some embodiments, the article for use with the non-combustible aerosol provision device may comprise aerosolizable material or an area for receiving aerosolizable material. In some embodiments, the article for use with the non-combustible aerosol provision device may comprise a mouthpiece. The area for receiving aerosolizable material may be a storage area for storing aerosolizable material. For example, the storage area may be a reservoir. In some embodiments, the area for receiving aerosolizable material may be separate from, or combined with, an aerosol generating area.

Thus there has been described a component for use in an aerosol provision system, the component comprising a region through which, in use, an aerosol from the aerosol provision system passes, the component being configured to enable selective interaction of the aerosol with a substance locatable in the region.

The aerosol provision system may be used in a tobacco industry product, for example a non-combustible aerosol provision system.

In one embodiment, the tobacco industry product comprises one or more components of a non-combustible aerosol provision system, such as a heater and an aerosolizable substrate.

In one embodiment, the aerosol provision system is an electronic cigarette also known as a vaping device.

In one embodiment the electronic cigarette comprises a heater, a power supply capable of supplying power to the heater, an aerosolizable substrate such as a liquid or gel, a housing and optionally a mouthpiece.

In one embodiment the aerosolizable substrate is contained in or on a substrate container. In one embodiment the substrate container is combined with or comprises the heater.

In one embodiment, the tobacco industry product is a heating product which releases one or more compounds by heating, but not burning, a substrate material. The substrate material is an aerosolizable material which may be for example tobacco or other non-tobacco products, which may or may not contain nicotine. In one embodiment, the heating device product is a tobacco heating product.

In one embodiment, the heating product is an electronic device.

In one embodiment, the tobacco heating product comprises a heater, a power supply capable of supplying power to the heater, an aerosolizable substrate such as a solid or gel material.

In one embodiment the heating product is a non-electronic article.

In one embodiment the heating product comprises an aerosolizable substrate such as a solid or gel material, and a heat source which is capable of supplying heat energy to the aerosolizable substrate without any electronic means, such as by burning a combustion material, such as charcoal.

In one embodiment the heating product also comprises a filter capable of filtering the aerosol generated by heating the aerosolizable substrate.

In some embodiments the aerosolizable substrate material may comprise an aerosol or aerosol generating agent or a humectant, such as glycerol, propylene glycol, triacetin or diethylene glycol.

In one embodiment, the tobacco industry product is a hybrid system to generate aerosol by heating, but not burning, a combination of substrate materials. The substrate materials may comprise for example solid, liquid or gel which may or may not contain nicotine. In one embodiment, the hybrid system comprises a liquid or gel substrate and a solid substrate. The solid substrate may be for example tobacco or other non-tobacco products, which may or may not contain nicotine. In one embodiment, the hybrid system comprises a liquid or gel substrate and tobacco.

In order to address various issues and advance the art, the entirety of this disclosure shows by way of illustration various embodiments in which the claimed invention(s) may be practiced and provide for a superior electronic aerosol provision system. The advantages and features of the disclosure are of a representative sample of embodiments only, and are not exhaustive and/or exclusive. They are presented only to assist in understanding and teach the claimed features. It is to be understood that advantages, embodiments, examples, functions, features, structures, and/or other aspects of the disclosure are not to be considered limitations on the disclosure as defined by the claims or limitations on equivalents to the claims, and that other embodiments may be utilized and modifications may be made without departing from the scope and/or spirit of the disclosure. Various embodiments may suitably comprise, consist of, or consist essentially of, various combinations of the disclosed elements, components, features, parts, steps, means, etc. In addition, the disclosure includes other inventions not presently claimed, but which may be claimed in future.

1. A component for use in an aerosol provision system, the component comprising:
a region through which, in use, an aerosol from the aerosol provision system passes, the component being configured to enable selective interaction of the aerosol with a substance locatable in the region.

2. The component according to claim 1, further comprising a guide element arranged to enable the selective interaction of the aerosol with the substance locatable in the region.

3. The component according to claim 2, wherein the guide element is arranged to be controllably movable.

4. The component according to claim 3, further comprising a controller arranged to receive a signal relating to movement of the guide element and arranged to control movement of the guide element.

5. The component according to claim 2, wherein the guide element comprises a plurality of movable elements, each of the plurality of movable elements arranged, in use, to move from a respective first position to a respective second position, wherein the respective first position allows the aerosol to flow past the movable element, and the respective second position prevents the aerosol from flowing past the movable element.

6. The component according to claim 5, wherein the plurality of movable elements are relatively movable with respect to one another.

7. The component according to claim 2, wherein the guide element is formed at least in part of a smart material.

8. The component according to claim 2, wherein the guide element is arranged substantially adjacent to the region.

9. The component according to claim 1, further comprising the substance located in the region.

10. An aerosol provision device comprising:
a first region configured for storage of an aerosol forming medium; and a second region configured for storage of a component comprising a substance region through which, in use, an aerosol formed from the aerosol forming medium passes, the aerosol provision device being configured to enable selective interaction of the aerosol with a substance locatable in the substance region.

11. The aerosol provision device according to claim 10, further comprising a guide element arranged to enable selective interaction of the aerosol with the substance locatable in the substance region.

12. The aerosol provision device according to claim 11, wherein the guide element is arranged to be controllably movable.

13. The aerosol provision device according to claim 12, further comprising a controller arranged to receive a signal relating to movement of the guide element and arranged to control movement of the guide element.

14. The aerosol provision device according to any of claims 11 to 13 claim 11, wherein the guide element is arranged substantially adjacent to the second region.

15. The aerosol provision device according to claim 10, further comprising the aerosol forming medium arranged in the first region.

16. An aerosol provision system comprising:
a first region storing an aerosol forming medium; and
a second region storing a component comprising a substance region through which, in use, an aerosol formed from the aerosol forming medium passes,
the aerosol provision system being configured to enable selective interaction of the aerosol with a substance locatable in the substance region.

17. The aerosol provision system according to claim 16, further comprising a guide element arranged to enable selective interaction of the aerosol with the substance locatable in the substance region.

18. The aerosol provision system according to claim 17, wherein the guide element is arranged to be controllably movable.

19. The aerosol provision device according to claim 18, further comprising a controller arranged to receive a signal relating to movement of the guide element and arranged to control movement of the guide element.

20. The aerosol provision system according to claim 17, wherein the guide element is arranged substantially adjacent to the second region.

21-23. (canceled)
